(19) United States
(12) Patent Application Publication
(10) Pub. No.: US 2024/0091142 A1
(43) Pub. Date: Mar. 21, 2024

(54) RESCUE COMPOSITIONS FOR HYPOGLYCEMIA

(71) Applicant: Empire Drying Services LLC, Newark, NJ (US)

(72) Inventors: David ROSENBERG, Monsey, NY (US); Michael WILHELM, Monsey, NY (US)

(21) Appl. No.: 18/370,572

(22) Filed: Sep. 20, 2023

(30) Foreign Application Priority Data
Sep. 21, 2022 (IL) 296690

(51) Int. Cl. A61K 9/00; A61K 9/20; A61K 31/7004; A61K 47/12; A61K 47/46

(52) U.S. Cl. CPC A61K 9/0056; A61K 9/2072; A61K 31/7004; A61K 47/12; A61K 47/46

(57) ABSTRACT

Provided herein are freeze-dried rescue compositions for hypoglycemia, that rapidly dissolve in the mouth, and processes for preparation thereof.

### CROSS REFERENCE TO RELATED APPLICATIONS

This application claims the benefit of priority of Israeli Patent Application No. 296690, filed Sep. 21, 2022, the contents of which is incorporated herein by reference in its entirety.

### TECHNOLOGICAL FIELD

The present invention provides rescue compositions for hypoglycemia, that rapidly dissolve in the mouth, and processes for preparation thereof.

### BACKGROUND

Hypoglycemia, also called low blood sugar, is a fall in blood sugar to levels below normal. Hypoglycemia may result in headache, tiredness, clumsiness, trouble talking, confusion, fast heart rate, sweating, shakiness, nervousness, hunger, loss of consciousness, seizures, or even death. Symptoms typically come on quickly.

The risk of hypoglycemia is greater in diabetics (with either diabetes type I or type II) who have eaten less than usual, recently exercised, or consumed alcohol. Other causes of hypoglycemia include severe illness, sepsis, kidney failure, liver disease, hormone deficiency, tumors such as insulinomas or non-B cell tumors, inborn errors of metabolism, several medications, and alcohol.

After hypoglycemia in a person is identified, rapid treatment is necessary and can be lifesaving. The main goal of treatment is to raise blood glucose back to normal levels, which is often done by administering a sugar, such as glucose. Treatment depends on the severity of symptoms and intravenous access.

If a patient is conscious and able to swallow safely, then food, drink, glucose sweets or glucose gel may be administered. However, oral administration may be a choking hazard, particularly if symptoms progress rapidly.

If a patient is confused, unresponsive or unconscious, intravenous dextrose is commonly administered, or when there is no intravenous access, intramuscular or intra-nasal glucagon may be administered. In the United States, glucacon kits are available by prescription for diabetic patients to carry in case of an episode of severe hypoglycemia. A glucagon kit delivers glucagon either by injection into a muscle or through nasal inhalation. However, non-medical personnel may have difficulty with or be reluctant to use such forms of administration.

Therefore, there is a need for an oral rescue composition for delivering a large quantity of sugar for immediate absorption to treat hypoglycemia.

### SUMMARY

According to some embodiments, provided herein are advantageous rescue compositions for hypoglycemia, that instantly melt in the mouth, and processes for preparation thereof.

Some embodiments relate to an orally dissolving composition including:
at least 85% w/w sugar;
about 5-10% w/w flavoring agent; and
about 0.1-5 w/w pH modifying agent,
wherein to the composition may be solid and/or may have a water content of less than about 5% w/w.

According to some embodiments, the sugar is a powdered sugar. Optionally, the sugar may be selected from dextrose, glucose, fructose, galactose, sucrose, lactose, maltose, and/or a combination thereof. Preferably, the sugar is dextrose.

According to some embodiments, the flavoring agent may be a natural flavoring, nature-identical flavoring, artificial flavoring, and/or a mixture thereof. Optionally, the flavoring agent may be a fruit or vegetable puree or concentrate, or combination thereof. Optionally, the flavoring agent may be a fruit puree or concentrate selected from cherry, pineapple, apple, pear, banana, orange, peach, raspberry, strawberry, blueberry, blackberry, nectarine, mandarin, kumquat, melon, watermelon, plumb, grape, grapefruit, or a combination thereof. Optionally, the flavoring agent may be a vegetable puree or concentrate selected from the group comprising: beet, sweet potato, pumpkin, carrot, corn, zucchini, potato.

According to some embodiments, the pH modifying agent may be selected from an edible acid or edible base. Optionally, the edible acid or edible base may be selected from citric acid, malic acid, acetic acid, tartaric acid, oxalic acid, caffeic acid, tannic acid, benzoic acid, lactic acid, folic acid, pantothenic acid, oleic acid, palmitic acid, sodium bicarbonate, magnesium hydroxide, aluminum hydroxide, ammonium bicarbonate, potassium bicarbonate, sodium hydroxide, calcium hydroxide, calcium carbonate, trisodium phosphate, sodium benzoate, and/or combinations thereof. Preferably, the edible acid may be citric acid, malic acid and/or a combination thereof.

According to some embodiments, the composition may further include an isotonicity adjusting agent. Optionally, the isotonicity adjusting agent may be selected from sodium chloride, sodium lactate, potassium chloride, mannitol, glycerin, and/or combinations thereof.

According to some embodiments, the orally dissolving composition may include:
about 0.1-1% w/w malic acid;
about 0.1-1% w/w citric acid;
about 1-7% w/w cherry puree;
about 1-7% w/w pineapple puree; and
about 85-95% w/w dextrose.

According to some embodiments, the composition may be configured to enable the sugar to be absorbed and/or melted by the oral mucosa.

According to some embodiments, the composition may be configured/designed to enable large quantity of the sugar to be immediately absorbed/melted by the oral mucosa without suffocation hazard.

According to some embodiments, the composition may take various forms, including but not limited to, an orally disintegrating (single) tablet, lozenges, troches, pellets, and thin films. Each possibility is a separate embodiment.

According to some embodiments, the composition doses may vary depending on the specific therapeutic need, and patient requirements, and can range from micrograms (μg) to milligrams (mg) or even grams (g).

According to some embodiments, the composition may be use in the treatment of hypoglycemia. According to some embodiments, the composition may be used as an energy booster. Some embodiments relate to a method for treating a hypoglycemic event including administering the composition to a patient in need thereof.

According to some embodiments, the sugar is the active ingredient of the composition. According to some embodiments, the sugar is the active ingredient of the composition for treating hypoglycemia and/or for using it as an energy booster.

Some embodiments relate to a process for producing an orally dissolving composition including:
mixing a composition comprising:
about 60-75% w/w sugar;
about 3-5% w/w flavoring agent; and
about 0.01-3% w/w pH modifying agent,
   with about 30-35% w/w water;
   flash freezing the mixture; and
   freeze drying the frozen mixture to a water content of less than 5% w/w.

According to some embodiments, the flash freezing may take places at a temperature in the range between about −60 to about −200° C. Optionally, the flash freezing may include flash freezing the mixture into frozen sheets. Optionally, the frozen mixture may be cut into pellets prior to freeze drying. Optionally, the pellets may be cut into strip, rectangular, square, triangular, circular, spherical, cylindrical, cubed, brick, and/or pyramidal shapes.

According to some embodiments, freeze drying the frozen mixture may reduce the water content to less than about 2% w/w.

According to some embodiments, the freeze-dried composition may be vacuum packaged.

Advantageously, the freeze-dried composition is a safely, ready to be consumed, non-powdered porous solid (also referred to herein as a “solid matrix” or “non-compressed solid”) comprising large quantity of sugar for immediate absorption by the oral mucosa. For a patient in need, buccal administration of the herein form of composition (i.e., freeze-dried non powdered solid matrix) is safe and easy, without the need for additional liquid, without suffocation/choking hazard and without the need of medical personnel.

Advantageously, the herein disclosed freeze-dried composition is an airy/porous non-compressed composition that was not formed by compressing or compacting powdered or granulated materials, which might sometimes lead to the degradation or loss of function of ingredients.

Advantageously, the herein disclosed freeze-dried composition is not highly friable and/or brittle, making it easy to pack and administer it as a whole and complete dosage.

Certain embodiments of the present disclosure may include some, all, or none of the above advantages. One or more technical advantages may be readily apparent to those skilled in the art from the figures, descriptions and claims included herein. Moreover, while specific advantages have been enumerated above, various embodiments may include all, some or none of the enumerated advantages.

In addition to the exemplary aspects and embodiments described above, further aspects and embodiments will become apparent by reference to the figures and by study of the following detailed descriptions.

### BRIEF DESCRIPTION OF THE FIGURES

The invention will now be described in relation to certain examples and embodiments with reference to the following illustrative figures so that it may be more fully understood.

In the Figures:

FIG. 1: A flowchart of steps in an exemplary process of preparing a hypoglycemic rescue composition, in accordance with some embodiments.

FIG. 2: A block diagram of an exemplary hypoglycemic rescue composition in accordance with some embodiments.

### DETAILED DESCRIPTION OF EMBODIMENTS

In the following description, various aspects of the disclosure will be described. For the purpose of explanation, specific configurations and details are set forth in order to provide a thorough understanding of the different aspects of the disclosure. However, it will also be apparent to one skilled in the art that the disclosure may be practiced without specific details being presented herein. Furthermore, well-known features may be omitted or simplified in order not to obscure the disclosure.

The present invention provides rescue compositions for hypoglycemia, that rapidly dissolve in the mouth, and processes for preparation thereof.

According to some embodiments, the composition may advantageously deliver a large quantity of a sugar rapidly for immediate absorption through the oral mucosa. According to some embodiments, the composition may rapidly dissolve and/or disintegrate in the mouth thereby alleviating the requirement to force a confused, unresponsive or unconscious person to swallow a food, drink, sweet or gel. Optionally, this may prevent and/or reduce the risk of choking. Optionally, the patient and/or a person assisting them need only slip the composition into the patients' mouth, therefore prior medical training is not required for administration.

Additionally, according to some embodiments, the composition may advantageously be a ready to be consumed product, which may rapidly dissolve and/or disintegrate in the mouth for quick absorption through the oral mucosa. Therefore, there is no need for swallowing and/or gastric absorption. Additionally, while the composition may be used sublingually, it is advantageously capable of being absorbed through any of the oral mucosa, and therefore may not require positioning under the tongue, either by a patient with hypoglycemic symptoms or by a lay-person who may be hesitant to do so. Further, intravenous or intra-muscular administration of a glucose solution may require training and/or medical knowledge, which most by-standers do not have. Furthermore, intranasal administration may block airways if not administered correctly. The compositions according to some embodiments therefore advantageously provide a much-needed method of administering a rescue composition for hypoglycemia.

Surprisingly, turning the powder into a porous solid suitable for buccal administration rather than for swallowing was achieved by hydrating, freezing and then drying by freeze drying.

As used herein, the term “porous solid” refers to a composition having a non-powdered and non-compressed integral structure. Typically the porous solid is in the form of a bite (typically having a diameter of about 2 cm), which can be held in the hand and entered into the mouth in its entirety (essentially without disintegrating), yet which readily dissolves in the mouth without requiring biting or swallowing thereof.

As used herein the terms “hypoglycemia” and “hypoglycemic” may be used interchangeably and may refer to low blood sugar, wherein there is a fall in blood sugar to levels below normal, typically below about 70 mg/dL (about 3.9 mmol/L).

As used herein, the term “orally dissolving” refers to dissolving and/or disintegrating in the mouth within seconds without the need for additional liquid.

As used herein, the term “oral mucosa” refers to the mucous membrane lining the inside of the mouth, including the buccal mucosa, labial mucosa, alveolar mucosa, ventral tongue surface, floor of the mouth, soft palate, attached gingiva, hard palate, and dorsal tongue surface.

According to some embodiments, the composition may be suitable for use as a food product. As used herein, the term “food product” may be understood to encompass a product that is consumed to provide nutrition to a subject. According to some embodiments, the food product may be in a solid form that is ready to be consumed by directly introducing to the mouth.

As used herein, the term “freeze-dried”, “lyophilization” and “cryodesiccation” may be used interchangeably, and may result in an orally dissolving and/or orally disintegrating solid form. As used herein, “freeze-drying” refers to a low temperature drying process that involves freezing the product, lowering pressure, followed by removal of ice (water) by sublimation. This is in contrast to drying by most conventional methods that evaporate water using heat or vacuum (e.g., no less than 7 Mbar) or both.

As used herein, the term “ready to be consumed product”, refers to a composition which does not have to be further prepared in any manner, e.g., it does not need to be cooked, rehydrated, steamed, mixed with a liquid (hot or cold), fried, microwaved, or any other form of preparation.

According to some embodiments, a rescue composition for hypoglycemia may include sugar, flavoring agent and pH modifying agent wherein the composition may be freeze dried to a water content of less than about 5% w/w. Optionally, the composition may not include a stabilizer, a preservative, an additive, etc. Optionally, the composition may be gluten free. Optionally, the composition may be lactose free. Optionally, the composition may be dairy free. Optionally, the composition may be vegan friendly.

According to some embodiments, the sugar may be a powdered sugar. Optionally, the particle size of the sugar may be in the range between about 3 μm to about 100 μm, between about 3 μm to about 75 μm, or between about 3 μm to about 50 μm. Optionally, 75% of the sugar crystals may be smaller than about 100 μm, smaller than about 75 μm, smaller than about 50 μm, or smaller than about 30 μm. Each possibility is a separate embodiment.

According to some embodiments, the composition may include sugar in an amount of more than about 80% w/w, more than about 85% w/w, more than about 90% w/w, or more than 95% w/w of the total freeze-dried composition. Each possibility is a separate embodiment.

According to some embodiments, the composition may include sugar in an amount in the range of about 55% w/w to about 80% w/w, about 60% w/w to about 75% w/w, or about 65% w/w to about 70% w/w of the total wet composition prior to freeze-drying. Each possibility is a separate embodiment.

According to some embodiments, the sugar may be selected from the group including: dextrose, glucose, fructose, galactose, sucrose, lactose, maltose, and/or combinations thereof. Preferably, the sugar is dextrose.

According to some embodiments, the flavoring agent may be natural flavorings, nature-identical flavorings, artificial flavorings, or mixtures thereof. According to some embodiments, the flavoring agent may be a concentrate (e.g., ginger, raspberry, maltol, glycerin, cocoa, licorice, vanillin, ethyl vanillin, etc.), extract (e.g., vanilla, lemon, lime, banana, cherry, butterscotch, brandy, rum, etc.), syrup (e.g., citric acid, sarsaparilla, cherry, etc.), essence, and/or aromatic oil (e.g., caraway, clove, lemon, spearmint, rose, peppermint, almond, anise, celery, cassia, cinnamon, clove, lemon, nutmeg, orange, rosemary, savory, basil, sweet marjoram, thyme, wintergreen, etc.). Optionally, the composition may include a flavor enhancer. Each possibility is a separate embodiment.

According to some embodiments, the flavoring agent may be a fruit and/or vegetable puree. According to some embodiments, the composition may in addition to, or as an alternative to the fruit puree include a vegetable puree. Therefore, in some embodiments, the composition may include a fruit puree and/or a vegetable puree.

According to some embodiments, the term “fruit puree” may refer to any type of fruit or combination of fruits that has been cooked, ground, pressed, blended and/or sieved until it reaches a consistency of a creamy paste or liquid. Optionally, the flavoring agent may be a fruit puree and/or concentrate selected from the group including: cherry, pineapple, apple, pear, banana, orange, peach, raspberry, strawberry, blueberry, blackberry, nectarine, mandarin, kumquat, melon, watermelon, plumb, grape, grapefruit, and/or a combination thereof. Each possibility is a separate embodiment.

According to some embodiments, the term “vegetable puree” may refer to any type of vegetable and/or combination of vegetables that has been cooked, ground, pressed, blended or sieved until it reaches a consistency of a creamy paste or liquid. Optionally, the flavoring agent may be a vegetable puree and/or concentrate selected from the group including: beet, sweet potato, pumpkin, carrot, corn, zucchini, potato, fennel, and/or a combination thereof. Each possibility is a separate embodiment.

According to some embodiments, the composition may include one or more flavoring agents in an amount of at least about 2% w/w, at least about 5% w/w, at least about 7% w/w, at least about 10% w/w, at least about 12% w/w, or at least about 15% w/w of the total freeze-dried composition. Each possibility is a separate embodiment.

According to some embodiments, the composition may include one or more flavoring agents in an amount in the range of about 2% w/w to about 30% w/w, about 2% w/w to about 20% w/w, about 5% w/w to about 15% w/w, or about 5% w/w to about 10% w/w of the total freeze-dried composition. Each possibility is a separate embodiment.

According to some embodiments, the composition may include one or more flavoring agents in an amount in the range of about 1% w/w to about 7% w/w, about 2% w/w to about 6% w/w, or about 3% w/w to about 5% w/w of the total wet composition prior to freeze drying. Each possibility is a separate embodiment.

According to some embodiments, the composition may include one or more pH modifying agents. According to some embodiments, the one or more pH modifying agents may be selected from an edible acid and/or edible base. Optionally, the edible acid may be selected from the group including: citric acid, malic acid, acetic acid, tartaric acid, oxalic acid, caffeic acid, tannic acid, benzoic acid, lactic acid, folic acid, pantothenic acid, oleic acid, palmitic acid, etc., and/or combinations thereof. Optionally, the edible base may be selected from the group including: sodium bicarbonate, magnesium hydroxide, aluminum hydroxide, ammonium bicarbonate, potassium bicarbonate, sodium hydroxide, calcium hydroxide, calcium carbonate, trisodium phosphate, sodium benzoate, etc. and/or combinations thereof. According to some embodiments, the one or more pH modifying agents may be an edible buffer.

According to some embodiments, the composition may include one or more pH modifying agents in an amount in the range of about 0.1% w/w to about 2% w/w, about 0.1% w/w to about 5% w/w, about 0.3% w/w to about 1% w/w, about 0.5% w/w to about 2% w/w, or about 0.5% w/w to about 5% w/w of the total freeze-dried composition. Each possibility is a separate embodiment.

According to some embodiments, the composition may include one or more pH modifying agents in an amount in the range of about 0.01% w/w to about 3% w/w, about 0.05% w/w to about 2% w/w, or about 1% w/w to about 1.5% w/w of the total wet composition prior to freeze-drying. Each possibility is a separate embodiment.

According to some embodiments, the composition may include an isotonicity adjusting agent. Optionally, the isotonicity adjusting agent is selected from the group including: sodium chloride, sodium lactate, potassium chloride, mannitol, glycerin, or combinations thereof. Each possibility is a separate embodiment. Optionally, the use of an isotonicity adjusting agent may increase the rate of dissolution and/or disintegration of the composition in the oral cavity. Optionally, the use of an isotonicity adjusting agent may increase the rate of absorption of the sugar by the oral mucosa.

According to some embodiments, the composition may include one or more isotonicity adjusting agents in an amount in the range of about 0.1% w/w to about 2% w/w, about 0.1% w/w to about 5% w/w, about 0.3% w/w to about 1% w/w, about 0.5% w/w to about 2% w/w, or about 0.5% w/w to about 5% w/w of the total composition. Each possibility is a separate embodiment.

According to some embodiments, the composition may include one or more isotonicity adjusting agents in an amount in the range of about 0.01% w/w to about 3% w/w, about 0.05% w/w to about 2% w/w, or about 1% w/w to about 1.5% w/w of the total wet composition prior to freeze-drying. Each possibility is a separate embodiment.

According to some embodiments, the composition contains less than 5% w/w water. According to some embodiments, the composition contains between about 0.1% w/w to about 5% w/w of water. According to some embodiments, the composition contains between about 0.5% w/w to about 5% w/w of water or about 1-3% w/w water. In some embodiments, the composition contains about 0.1% w/w, about 0.5% w/w, about 1% w/w, about 2% w/w, about 3% w/w, about 4% w/w, or about 5% w/w of the total freeze-dried composition. Each possibility is a separate embodiment.

According to some embodiments, during the process for preparing the composition, water in the range between about 20% w/w to about 50% w/w, between about 25% w/w to about 45% w/w, or between about 30% w/w to about 40% w/w of the wet mixture may be added to the other components of the composition. Each possibility is a separate embodiment.

According to some embodiments, a process for an orally dissolving composition may include:
mixing about 60-75% w/w sugar, about 3-5% w/w flavoring agent, and about 0.01-3% w/w pH modifying agent, with about 30-35% w/w water;
   flash freezing of the mixture; and
   freeze drying the frozen mixture to a water content of less than 5% w/w.

According to some embodiments, blending and/or mixing the ingredients may be performed using a high shear blender, agitator, high-speed disperser, rotor mixers, or stator mixer.

According to some embodiments, the mixture may be poured into sheets prior to flash freezing. Optionally, the mixture may be poured into one or more molds prior to flash freeing. Optionally, the mixture may be poured into an automatic molding machine e.g., a nitrogen freezing automatic molding machine. Optionally, the molded mixture may be deep frozen in a freezing tunnel.

According to some embodiments, the flash freezing may take place at a temperature in the range between about −60 to about −200° C., between about −70 to about −150° C., or between about −75 to about −100° C.

According to some embodiments, the frozen mixture may be cut and/or perforated into pellets prior to freeze drying. Optionally, the pellets may be strips, rectangles, squares, triangles, circles, spheres, cylinders, cubes, bricks, blocks, pyramids, etc. Optionally, the mixture may be poured into molds to produce pellets. Optionally, the pellets may be connected by seams to form a sheet. Optionally, the sheet of attached pellets may be broken apart into individual pellets. For example, the frozen sheets may be perforated into rectangular shapes of about 2.5 cm×3.8 cm and then broken into the pellets into the about 2.5 cm×3.8 cm shape by a breaking machine.

According to some embodiments, the freezing-drying may take place at a temperature in the range between about −5 to about −100° C., between about −10 to about −75° C., or between about −20 to about −50° C. According to some embodiments, the freezing-drying may take place at a pressure in the range between about 5 Pa to about 100 Pa, between about 10 Pa to about 50 Pa, or between about 20 Pa to about 30 Pa. According to some embodiments, the pellets may be freeze-dried for about 0.5 hour, about 1 hour, about 3 hours, about 6 hours, about 9 hours, about 12 hours, about 15 hours, about 18 hours, about 21 hours, about 24 hours, about 36 hours, about 48 hours, or about 72 hours.

According to some embodiments, the freeze-dried pellets may be packaged. Optionally, the freeze-dried pellets may be vacuum packaged. Optionally, the freeze-dried pellets may be packaged in a flow wrap machine. Optionally, the freeze-dried pellets may be packaged for easy access when needed. Optionally, the freeze-dried pellets may be packaged individually. Optionally, the freeze-dried pellets may be packaged in sets and/or kits.

According to some embodiments, an individual serving of the freeze-dried composition may deliver between about 20 g to about 100 g, or between about 25 g to about 50 g sugar. Optionally, the composition may disperse rapidly (in less than 25 s) in the mouth without requiring water. Advantageously, the composition may provide better pre-gastric absorption for the sugar and/or more efficient delivery of the sugar. Additionally, the composition may be ideal for administration to populations which suffer from dysphagia, require fast onset, and ease of use.

According to some embodiments, the composition may be suitable for use in the treatment of a hypoglycemia. According to some embodiments, the composition may be suitable for use as an energy booster. According to some embodiments, the composition may include one or more therapeutic compounds.

According to some embodiments, there is provided a method for administering a sugar to a subject in need thereof, the method comprising inserting into the subject's mouth, the herein disclosed composition.

Reference is now made to the figures.

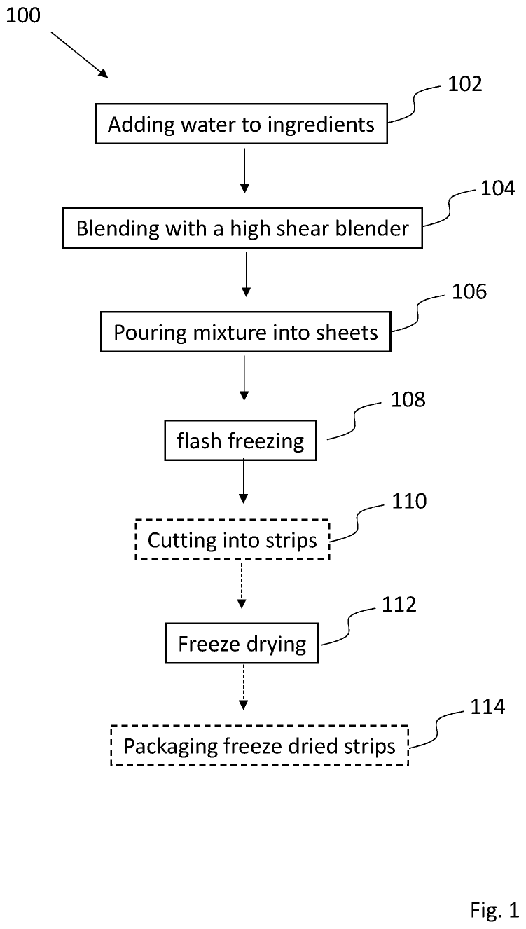

FIG. 1 is a flowchart of steps in an exemplary process of preparing a hypoglycemic rescue composition, in accordance with some embodiments. For example, in process 100, water is added 102 to the other ingredients (e.g., sugar, flavor agent, pH modifier, isotonicity agent, etc.) and mixed 104 in a high shear blender. The mixture is then poured 106 into a sheet or mold and flash frozen 108, e.g., by nitrogen. The mixture is molded into pellets connected by seams which are broken into individual pellets, perforated sheets which may be broken into individual pellets and/or frozen into sheets which may be cut 110 into strips or pellets. Optionally, the flash frozen mixture is then deep frozen in a freezing tunnel. The frozen mixture is then freeze dried 112 to remove the added water, and any other moisture from the other ingredients. The freeze-dried composition is then packaged 114.

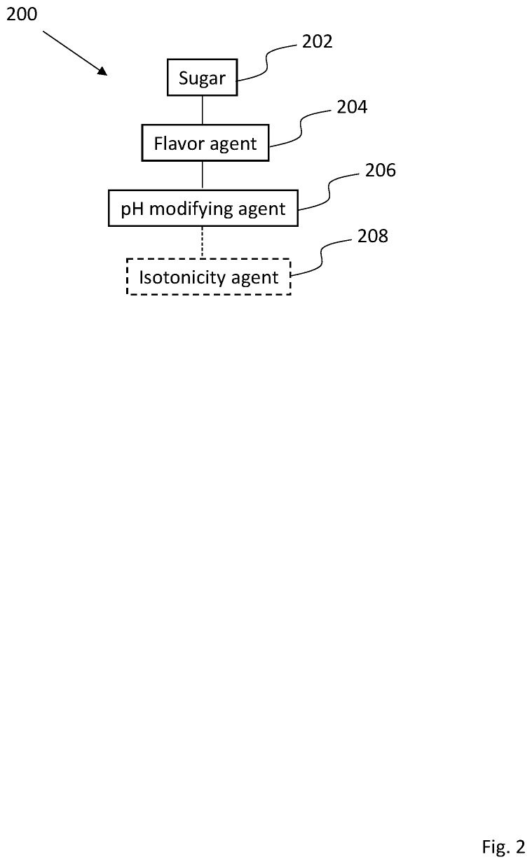

FIG. 2 is a block diagram of an exemplary hypoglycemic rescue composition in accordance with some embodiments. For example, the composition 200, may include a sugar 202 (e.g., dextrose, glucose, fructose, galactose, sucrose, lactose, maltose, etc.), one or more flavor agents 204 (e.g., a fruit puree, vegetable puree and/or a combination thereof), a pH modifier 206 (e.g., one or more edible acids or one or more edible bases), and optionally, an isotonicity agent 208 (e.g., sodium chloride, sodium lactate, potassium chloride, mannitol, glycerin, or combinations thereof).

The term “a” and “an” refers to one or to more than one (i.e., to at least about one) of the grammatical object of the article. By way of example, “an element” means one element or more than one element.

The term “about” when referring to a measurable value such as an amount, a temporal duration, and the like, is meant to encompass variations of ±20% or in some instances ±10%, or in some instances ±5%, or in some instances ±1%, or in some instances ±0.1% from the specified value, as such variations are appropriate to perform the disclosed methods.

The term “consisting essentially of” means that the composition, method or structure may include additional ingredients, steps and/or parts, but only if the additional ingredients, steps and/or parts do not materially alter the basic and novel characteristics of the claimed composition, method or structure. The following examples are presented in order to more fully illustrate some embodiments of the invention. They should in no way be construed, however, as limiting the broad scope of the invention. One skilled in the art can readily devise many variations and modifications of the principles disclosed herein without departing from the scope of the invention.

### EXAMPLES

### Example 1: Process for the Preparation of an Orally Dissolving Rescue Composition for Hypoglycemia

| | Amount (% w/w of |
|---|---|
| Ingredient | dry composition) |
| Malic Acid | 0.43 |
| Citric Acid | 0.43 |
| Cherry puree | 4.73 |
| Pineapple Puree | 2.03 |
| Dextrose | 92.39 |

In order to achieve required freeze-dried texture, water is added in an amount of 33% w/w of the wet composition. The water is lost during the freeze-drying process and the above ingredients and ratios remain.

Production process: 1) Mix the ingredients with the water in a high shear blender. 2) mix until a smooth consistency is achieved. 3) Pour the mixture into sheets. 4) Flash freezing into frozen sheets perforated into rectangular shapes of about 2.5 cm×3.8 cm. 3) Break the pellets into the about 2.5 cm×3.8 cm shape. 4) Place pellets into freeze dryer for 24 hours. 5) Package in flow wrap machine.

While certain embodiments of the invention have been illustrated and described, it will be clear that the invention is not limited to the embodiments described herein. Numerous modifications, changes, variations, substitutions and equivalents will be apparent to those skilled in the art without departing from the spirit and scope of the present invention as described by the claims, which follow.

1. An orally dissolving composition comprising:
at least about 85% w/w sugar;
about 5-10% w/w flavoring agent; and
about 0.1-5% w/w pH modifying agent,
wherein the composition is an orally dissolving, freeze dried, non-powdered solid matrix having a water content of about 0.5-5% w/w.

2. The composition of claim 1, wherein the sugar is a powdered sugar.

3. The composition of claim 1 or claim 2, wherein the sugar is selected from dextrose, glucose, fructose, galactose, sucrose, lactose, maltose, or a combination thereof.

4. The composition of claim 3, wherein the sugar is dextrose.

5. The composition of claim 1, wherein the flavoring agent is a natural flavoring, nature-identical flavoring, artificial flavoring, or a mixture thereof.

6. The composition of claim 1, wherein the flavoring agent is a fruit or vegetable puree or concentrate, or combination thereof.

7. The composition of claim 6, wherein the flavoring agent is a fruit puree or concentrate selected from cherry, pineapple, apple, pear, banana, orange, peach, raspberry, strawberry, blueberry, blackberry, nectarine, mandarin, kumquat, melon, watermelon, plum, grape, grapefruit, or a combination thereof.

8. The composition of claim 6, wherein the flavoring agent is a vegetable puree or concentrate selected from the group comprising: beet, sweet potato, pumpkin, carrot, corn, zucchini, potato.

9. The composition of claim 1, wherein the pH modifying agent is selected from citric acid, malic acid, acetic acid, tartaric acid, oxalic acid, caffeic acid, tannic acid, benzoic acid, lactic acid, folic acid, pantothenic acid, oleic acid, palmitic acid, sodium bicarbonate, magnesium hydroxide, aluminum hydroxide, ammonium bicarbonate, potassium bicarbonate, sodium hydroxide, calcium hydroxide, calcium carbonate, trisodium phosphate, sodium benzoate, or combinations thereof.

10. The composition of claim 9, wherein the edible acid is citric acid, malic acid or a combination thereof.

11. The composition of claim 1, further comprising an isotonicity adjusting agent.

12. The composition of claim 11, wherein the isotonicity adjusting agent is selected from: sodium chloride, sodium lactate, potassium chloride, mannitol, glycerin, or combinations thereof.

13. The composition of claim 1 comprising:
about 0.1-1% w/w malic acid;
about 0.1-1% w/w citric acid;
about 1-7% w/w cherry puree;
about 1-7% w/w pineapple puree; and
about 85-95% w/w dextrose.

14. The composition of claim 1, wherein the composition enables the sugar to be absorbed by the oral mucosa.

15. The composition of claim 1, for use in the treatment of hypoglycemia or as an energy booster.

16. The composition of claim 1, consisting essentially of at least about 85% w/w sugar; about 5-10% w/w flavoring agent; and about 0.1-5% w/w pH modifying agent.

17. A process for producing an orally dissolving composition comprising:
mixing a composition comprising:
about 60-75% w/w sugar;
about 3-5% w/w flavoring agent; and
about 0.01-3% w/w pH modifying agent,
with about 30-35% w/w water;
flash freezing the mixture; and
freeze drying the frozen mixture to a water content of about 0.5-5% w/w to obtain ready to consume, orally dissolving, non-powdered porous solid comprising at least about 85% w/w sugar.

18. The process of claim 17, further comprises cutting the frozen mixture into pellets prior to freeze drying.

19. The process of claim 17, further comprising vacuum packaging the freeze-dried composition.

20. The process of claim 17, wherein the freeze drying of the frozen mixture reduces the water content to less than about 2% w/w.
